(19) United States
(12) Patent Application Publication
(10) Pub. No.: US 2009/0092591 A1
(43) Pub. Date: Apr. 9, 2009

(54) Use of a Mixture of Superoxide Dismutase and Catalase for Treating Inflammatory Skin Lesions

(76) Inventor: Christian Diehl, Cordoba (AR)

Correspondence Address: OLIFF & BERRIDGE, PLC, P.O. BOX 320850, ALEXANDRIA, VA, 22320-4850, US

(21) Appl. No.: 11/988,053

(22) PCT Filed: Jun. 28, 2005
(86) PCT No.: PCT/IB2005/001837

§ 371 Date: Apr. 25, 2008

(51) Int. Cl. A61K 38/44

(52) U.S. Cl. 424/94.2

(57) ABSTRACT

The invention concerns the use of a mixture of superoxide dismutase and catalase for preparing a topical composition for treating inflammatory skin lesions, said lesions being selected among the group consisting of dermal fibroses, epidermal keratoses, cheloid scars or hypertrophic eschars. The inventive compositions are formulated to obtain all galenic forms conventionally used for applying a topical composition on the skin such as for example milk, cream, lotion, plaster or patch, as powder sticks to be solubilized in water or physiological serum prior to use.

Use of a mixture of superoxide dismutase and catalase for treating inflammatory skin lesions.

Among the lesions of the dermis or epidermis, which are sometimes incapacitating, there are some which at present are still lacking truly satisfactory treatment in spite of numerous attempts that have been carried out: these lesions are dermal fibroses, epidermal keratoses, keloids or hypertrophic scars.

Although these lesions have neither the same causes nor the same genesis, they nevertheless do involve similar mechanisms, which have allowed a composition to be developed for the manufacture of a dermal or dermocosmetic formulation intended for the treatment of these complaints and more particularly keloids and cutaneous fibroses, especially radio-induced fibroses.

Fibroses are non-specific lesions which are characterized by hyperplasia of the conjunctive tissues with proliferation of the fibroblasts or of the fibrocytes, forming collagen. They give rise to conversion into fascicules composed primarily of conjunctive tissue, subsequent to a pathological phenomenon or a therapeutic act.

The activation of the fibroblasts is often cited as one of the causes of the origin of this pathology. In a normal cicatrization process, the fibroblasts are temporarily activated to become myofibroblasts, to proliferate and to form the collagen matrix.

A part of the skin that is affected by fibrosis may, in a certain way, be considered to be a region in which the signals which activate cicatrization are emitted continuously, thus giving rise to abnormal production of cytokines and growth factors, this abnormal production producing chronic activation and, in the long term, myofibroblasts, which then give fibrosis.

The known triggering factors include radiation, especially radiotherapy, and free radicals, and may be potentialized by chemotherapeutic treatments such as bleomycin.

Additionally, the interaction of ionizing radiation with living tissue, via the production of free radicals, initiates biological disorders and damage which produce an inflammatory reaction.

A great number of hypotheses have been expressed on the involvement of various phenomena, such as the involvement of TGF-β1 factors, and on possible treatments, such as the use of treatments based on halofuginone.

To date, however, there is no satisfactory treatment in existence.

The keloid is a benign skin tumor which in general terms has the appearance of a fold and which develops on scars. It is caused by the uncontrolled growth of fibrous tissue which develops beyond the cicatrization region and does not retreat following excision. This type of disregulation of the cicatrization process leads or may lead also to the hypertrophic scars which appear when there is a disequilibrium between the anabolic and catabolic phases of the cicatrization process, giving rise to the production of collagen in an amount greater than the amount which is degraded, and to the growth of the scar in all directions.

The essential difference between keloids and hypertrophic scars appears only when the lesions are mature, the histological difference essentially being the presence of keloid collagen in the keloids. At the start of their formation, an inflammatory phase appears, often in the form of erythema. The treatments are varied and are essentially preventive: they include dressings, compression during cicatrization, corticosteroids, radiotherapy (very controversial), and interferon-based treatments.

Surgical interventions are also widely practiced, as are cryosurgery and laser treatment. Therapeutic attempts based on the administration of active molecules which affect the synthesis of the collagen have been carried out, said molecules including proline-6-hydroxyproline, azetidinecarboxylic acid, trinilast or molecules having antiallergic activity. The use of growth factors has also given more or less satisfactory results.

In addition, it is apparent that the disorders of the cicatrization process which lie beneath these different pathologies may have an inflammatory origin which is due to oxidative processes.

Pharmaceutical compositions for preventing and treating the damage caused by oxidation in the phases of reperfusion following cardiac surgery, an ischemic accident or blood clots, based on superoxide dismutase (SOD) and catalase (CAT), are known from U.S. Pat. No. 5,080,886.

Furthermore, a clinical trial has confirmed the advantage of topical application of SOD in the reduction of post-irradiation fibrosis in cancer of the breast in 2004: see Campana et al., J. Cell. Med., vol. 8, No. 1, 2004, 109-116.

However, this advantage was not confirmed in the treatment of the other lesions of inflammatory origin cited above.

The present invention accordingly relates to the use of a mixture of superoxide dismutase and catalase for preparing a topical composition intended for the treatment of dermal fibroses, epidermal keratoses, keloids or hypertrophic scars.

Superoxide dismutase (SOD) is a stable enzyme of natural origin: it gives out superoxide radicals without being consumed, and it is generally soluble in water. Superoxide dismutase is either of plant origin or obtained by biotechnology.

Catalase is a ferriporphyrin enzyme that catalyzes the liberation of molecular oxygen from hydrogen peroxide.

These enzymes are referred to as metabolic enzymes, which catalyze reactions within the cell, such as the production of energy and detoxification. In the natural state these enzymes are frequently present simultaneously, since their mechanisms are complementary.

Catalase protects the cell by attacking the free superoxide radicals.

There are three types of SOD: copper/zinc SOD, manganese SOD, and ferric SOD. These enzymes protect the cells: SOD Cu/Zn protects the cytoplasm, where the free radicals are produced by metabolic reactions, and SOD Mn protects the mitochondria of the cell, that contains the genetic information and acts as the site for the production of cell energy.

In the formulations according to the invention, the SOD may be of any origin.

SOD is naturally present in the majority of plants: it is found in extracts, apples, certain varieties of cabbage, broccolis, Brussels sprouts, or even cabbage or melon, which are optionally transgenic, and also horseradish, or it may be extracted from seeds or shoots of enzyme-rich cereals, such as wheat, corn, soybean or barley.

It is also produced by biotechnology; for example, it may originate from a strain of Saccharomyces cerevisiae.

It is potentially extracted from bovine or human erythrocytes, produced by recombinant synthesis by microorganisms such as E. coli or yeasts, or else extracted from mammalian livers.

The invention accordingly relates in one embodiment to a composition characterized in that the superoxide dismutase is obtained by biotechnology, originating for example from a natural strain of Saccharomyces cerevisiae.

According to the invention the SOD may be complexed or bonded to polymers without prejudice to its enzymatic activity—for example, polyethylene glycol or polysaccharides.

The catalase (CAT) which converts hydrogen peroxide to water and oxygen may be of any origin.

The catalase may, for example, be obtained from mammalian liver extract or from microorganisms such as Aspergillus niger.

It may also be obtained from plant extracts or obtained by recombinant synthesis.

Like the SOD, it may be bonded covalently or complexed to polymers without prejudice to its enzymatic activity—for example, polyethylene glycol or polysaccharides.

These two enzymes may be encapsulated or incorporated in polymeric microparticles, composed for example of crosslinked ionic polysaccharides and/or hydrophilic polymers.

These formulations of these two enzymes, alone or as a mixture, allow the enzymatic activity to be protected, for example, from interactions with the outside environment, while promoting targeting, spreading, and also pharmaceutical formulation.

These two enzymes may be present in a lyophilized form, powder for example, in crystalline suspension form or in ammonium sulfate suspension form.

Numerous animal or plant extracts contain these two enzymes in association: examples include melon extract, cabbage extract or mammalian liver extract.

According to the invention the SOD/CAT mixture, whether original, meaning that the extract contains the mixture of the two enzymes on extraction, or whether prepared, must allow an enzymatic activity ratio of between 14/2 and 15/5, with an enzymatic SOD activity of between 10 000 and 16 000 IU per gram.

The activity of the catalase will be between approximately 1500 and 4000 IU per gram.

The enzymatic activity of the SOD is determined by the method of Beauchamp C. and Fridovich I., Analytical Biochemistry 44, 276 (1971), modified by the method of Spitz, D., and Oberley, L.: An Assay for Superoxide Dismutase Activity in Mammalian Tissue Homogenates, Anal. Biochem. 179, 8, 1989.

This method is an indirect method, owing to the short half-life of SOD; it allows the SOD to be evaluated via its capacity to inhibit a flow of superoxide anion generated by the xanthine/xanthine oxidase system.

The enzymatic activity of the catalase is determined by the method of ClaiBorne A., Catalase activity, CRC Handbook of Methods for oxygen radical research, 283-284, 1985.

This method is based on the decomposition of hydrogen peroxide by catalase, which is monitored using a UV spectrophotometer at 240 nm.

The invention accordingly relates to the use of a mixture of superoxide dismutase and catalase for preparing a topical composition intended for the treatment of skin lesions of inflammatory origin.

It relates to the use described above, characterized in that the lesions are selected from the group consisting of dermal fibroses, epidermal keratoses, keloids or hypertrophic scars.

It relates to the use described above, characterized in that the superoxide dismutase is extracted from apples, broccolis, Brussels sprouts, cabbage or melon, which are optionally transgenic, or horseradish.

It relates to the use described above, characterized in that the superoxide dismutase is extracted from seeds or shoots of enzyme-rich cereals selected from wheat, corn, soybean, and barley.

It relates to the use described above, characterized in that the superoxide dismutase is obtained by biotechnology.

It relates to the use described above, characterized in that the superoxide dismutase is obtained from a strain of Saccharomyces cerevisiae.

It relates to the use described above, characterized in that the superoxide dismutase is extracted from bovine or human erythrocytes or extracted from mammalian livers.

It relates to the use described above, characterized in that the superoxide dismutase is produced by recombinant synthesis by E. coli microorganisms or yeasts.

It relates to the use described above, characterized in that the superoxide dismutase is produced by biotechnology, by a natural strain of Saccharomyces cerevisiae.

It relates to the use described above, characterized in that the catalase is obtained from mammalian liver extracts or extracts of microorganisms such as Aspergillus niger.

It relates to the use described above, characterized in that the catalase is obtained from plant extracts.

It relates to the use described above, characterized in that the catalase is obtained by recombinant synthesis.

It relates to the use described above, characterized in that the catalase or the superoxide dismutase, alone or as a mixture, are bonded covalently or complexed to polymers.

It relates to the use described above, characterized in that the polymers are selected from polyethylene glycol and polysaccharides.

It relates to the use described above, characterized in that the catalase or the superoxide dismutase, alone or as a mixture, are encapsulated or incorporated in polymeric microparticles.

It relates to the use described above, characterized in that the polymeric microparticles are composed of crosslinked ionic polysaccharides and/or of hydrophilic polymers.

It relates to the use described above, characterized in that the catalase and the superoxide dismutase, alone or as a mixture, are in a lyophilized powder form, in crystalline suspension form or in ammonium sulfate suspension form.

It relates to the use described above, characterized in that the catalase and the superoxide dismutase are present as a mixture in a natural extract.

It relates to the use described above, characterized in that the natural extract is selected from melon extracts, cabbage extracts or mammalian liver extracts.

It relates to the use described above, characterized in that the SOD/CAT mixture exhibits an enzymatic activity ratio of between 14/2 and 15/5 with a SOD enzymatic activity of between 10 000 and 16 000 IU per gram.

It relates to the use described above, characterized in that the catalase activity is between approximately 1500 and 4000 IU per gram.

The invention is illustrated for example with the following formulation:

### FORMULATION EXAMPLES

Creams for local external application to the lesions.

| | |
|---|---|
| Propylene glycol dipelargonate | 6% |
| Propylene glycol | 5% |
| Glyceryl stearate and PEG stearate | 3% |
| Stearic acid | 3% |
| Xanthan gum | 2% |
| Ethylene glycol | 1% |
| Cetyl palmitate | 1% |
| Petrolatum | 0.5% |
| Avocado oil | 1% |
| Liquid paraffin | 2% |
| Triethanolamine | 0.67% |
| Potassium sorbate | 0.2% |
| Preservatives | 0.3% |
| Essential oils (neroli) | |
| SOD/CAT mixture | 2% |
| Water | qs |

The SOD/CAT mixture used is a mixture extracted from a variety of Brassica napus.

The enzymatic activity of the SOD is evaluated by the method described above as being approximately 14 000 IU per gram.

The enzymatic activity of the catalase by the method described above is evaluated as being 3000 IU.

In another example the SOD/CAT mixture is provided by a melon extract: the stabilized extract sold under the brand name Extramel by the company Bionov.

The enzymatic activity of the SOD is evaluated by the method described above as being approximately 14 000 IU per gram.

The enzymatic activity of the catalase by the method described above is evaluated as being 2000 IU.

In another example the SOD/CAT mixture is provided by a mixture comprising SOD and catalase which are obtained from plant extracts sold by the company Biotics Research Corporation.

The enzymatic activity of the SOD is evaluated by the method described above as being approximately 14 500 IU per gram.

The enzymatic activity of the catalase by the method described above is evaluated as being 2500 IU.

In another example the SOD/CAT mixture is provided by a mixture prepared in accordance with the final enzymatic activity required, namely 14 000 IU per gram for SOD and 3000 IU per gram for catalase, of SOD extracted from Escherichia coli in the form of lyophilized powder (2500 IU/mg) and from catalase extracted from Aspergillus niger in lyophilized form (170 IU/mg).

The compositions according to the invention are formulated to give all of the pharmaceutical forms that are conventionally used for the topical application of a composition to the skin.

They are formulated, for example, in the form of a milk, cream, lotion, plaster or patch, or in stick form, or even as a powder to be dissolved in water or physiological serum prior to use.

The compositions according to the invention may optionally contain various additives, such as suspension agents, emulsifiers, anionic, cationic, nonionic or amphoteric polymers, proteins, vitamins, surfactants, mineral oils, vegetable oils, waxes, silicone resins and/or rubbers, thickeners, acidifying or alkalifying agents, solvents, pH stabilizers, UV protectants, preservatives, antibacterial agents, and antifungal agents, fragrances or other adjuvants which are commonly used in cosmetology or dermatology.

1. A method for preparing a topical composition intended for the treatment of skin lesions of inflammatory origin comprising mixing (a) superoxide dismutase, extracted from apples, broccolis, Brussels sprouts, cabbage or melon, which are optionally transgenic, or horseradish, or extracted from seeds or shoots of enzyme-rich cereals selected from wheat, corn, soybean, and barley, and (b) catalase.

2. The method according to claim 1, wherein the lesions are selected from the group consisting of dermal fibroses, epidermal keratoses, keloids or hypertrophic scars.

3. The method according to claim 1, wherein the catalase is catalase obtained from mammalian liver extracts or extracts of microorganisms.

4. The method according to claim 1, wherein the catalase is catalase obtained from plant extracts.

5. The method according to claim 1, wherein the catalase is catalase obtained by recombinant synthesis.

6. The method according to claim 1, wherein the catalase and the superoxide dismutase, alone or as a mixture, are bonded covalently or complexed to polymers.

7. The method according to claim 6, wherein the polymers are selected from polyethylene glycol and polysaccharides.

8. The method according to claim 1, wherein the catalase and the superoxide dismutase, alone or as a mixture, are encapsulated or incorporated in polymeric microparticles.

9. The method according to claim 8, wherein the polymeric microparticles are composed of at least one member selected from the groups consisting of crosslinked ionic polysaccharides and hydrophilic polymers.

10. The method according to claim 1, wherein the catalase or the superoxide dismutase, alone or as a mixture, are in a lyophilized powder form, in crystalline suspension form or in ammonium sulfate suspension form.

11. The method according to claim 1, wherein the catalase and the superoxide dismutase are present as a mixture in a natural extract.

12. The method according to claim 1, wherein the natural extract is selected from melon extracts, cabbage extracts and mammalian liver extracts.

13. The method according to claim 1, wherein the mixture exhibits an enzymatic activity ratio of between 14/2 and 15/5 with a superoxide dismutase enzymatic activity of between 10,000 and 16,000 IU per gram.

14. The method according to claim 1, wherein a catalase activity of said mixture is between approximately 1500 and 4000 IU per gram.

15. The method according to claim 1, wherein the catalase is a catalase obtained from Aspergillus niger.
